(19) United States
(12) Patent Application Publication
(10) Pub. No.: US 2023/0381030 A1
(43) Pub. Date: Nov. 30, 2023

(54) TAMPON WITH NATURALLY HYDROPHOBIC COMPONENTS

(71) Applicant: The Procter & Gamble Company, Cincinnati, OH (US)

(72) Inventor: Khalid QURESHI, Mason, OH (US)

(21) Appl. No.: 17/751,970

(22) Filed: May 24, 2022

(51) Int. Cl. A61F 13/34; A61F 13/20; A61L 15/40; A61L 15/52; D06M 11/38; D06M 11/76

(52) U.S. Cl. CPC A61F 13/34; A61F 13/2085; A61L 15/40; A61L 15/52; D06M 11/38; D06M 11/76; A61F 2013/15243

(57) ABSTRACT

A tampon comprising an absorbent pledget and a withdrawal cord attached to the pledget is disclosed. The withdrawal cord may be constituted predominantly (by weight) of natural plant fiber such as cotton, and comprise no more than 5 percent by weight material derived from petroleum, while exhibiting limiting wicking capability. A method for manufacturing tampons with withdrawal cords including cotton fiber is disclosed, including a scouring step in which the scouring solution is of relatively lower pH than is conventional for scouring cotton, is disclosed.

### BACKGROUND

In recent years several trends in consumer preferences have influenced the designs of consumer products used for personal care and hygiene. Increasing numbers of consumers are exhibiting preferences for products they deem to be “natural”, which they may associate with being more healthful. Increasing numbers of consumers are also exhibiting preferences for products which they deem to be “sustainable”, which may be deemed to include several factors, generally including but not limited to raw material supply sustainability and minimized negative impact on the environment.

Additionally, regulatory pressures in some regions, discouraging use of plastics and/or other petroleum-derived materials as components of consumer products, are increasing.

These trends and pressures have impacted products including feminine hygiene products such as tampons.

An essential component of a tampon is a withdrawal cord. The withdrawal cord may be a length of string or yarn composed of fibers that are twisted, braided or knitted, attached to the tampon pledget by any suitable construction mechanism. The fibers may be any suitable combination of synthetic and/or natural fibers typically used or suitable as components of string or yarn, which are otherwise safe for the intended use as a tampon component.

A necessary feature of the withdrawal cord is that it resist wicking of menstrual fluid. Users do not want menstrual fluid to wick down the cord while the tampon is in use, which can create or increase a risk of soiling of underwear and outer garments.

Since menstrual fluid is generally aqueous, a cord formed of fibers having hydrophobic surface chemistry will tend to resist wicking of the fluid. For this reason, tampon withdrawal cords are frequently manufactured of yarns composed of fibers spun from polymer resins that will have inherently hydrophobic surfaces. Alternatively, constituent fibers, or a cord of constituent fibers, that are not deemed sufficiently hydrophobic, may be treated to render the fibers hydrophobic and/or to increase their hydrophobicity.

The market demand for products having “natural” components has led manufacturers of tampons to provide withdrawal cords with the tampons, that have in partial, predominant or substantially entire proportion, cotton and/or other plant fiber components.

Following harvesting and removal of seeds, leaves, stems and bodies of foreign matter by mechanical processes, cotton fibers are typically further processed for commercial use as components of medical products (such as tampons) via scouring and bleaching. These steps remove materials from the fibers including waxes, contaminant materials such as environmental pollutants, pesticides, herbicides, pollens, molds, yeasts, insect residues and allergen proteins, and also leave the fibers with a relatively bright white color.

Conventionally processed cotton fibers, however, have inherently highly hydrophilic surfaces. A yarn or string composed of conventionally processed cotton fibers will readily wick aqueous fluid such as menstrual fluid, unless mitigating steps and/or treatment are provided. For this reason, tampon manufacturers desiring to include a cotton-based withdrawal cord are currently compelled to treat the cord material with an agent to render the cotton fiber hydrophobic. Agents such as various waxes (such as paraffin, derived from petroleum) are typically applied for this purpose.

Such treatment, however, adds processing steps, and energy and material costs, to production of the cord. Further, adding components derived from petroleum to the cord material is counterproductive to the goal of decreasing use of/reliance on petroleum.

Accordingly, there is room for improvement in tampon cords, their component materials and methods of production, in response to trending consumer preferences and regulatory pressures, while still delivering satisfactory products.

### DESCRIPTION OF THE DRAWINGS

FIG. 1 is a perspective view of a tampon with its pledget in a compressed form.

FIG. 2 is a perspective view of an example of a tampon prior to compression of its pledget.

FIG. 3 is a perspective view of another example of a tampon prior to compression of its pledget.

FIG. 4 is a schematic view of an arrangement of equipment and a test specimen, for the Withdrawal Cord Wicking Height measurement method described herein.

### DESCRIPTION OF EXAMPLES

### Definitions

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

Tampon Construction

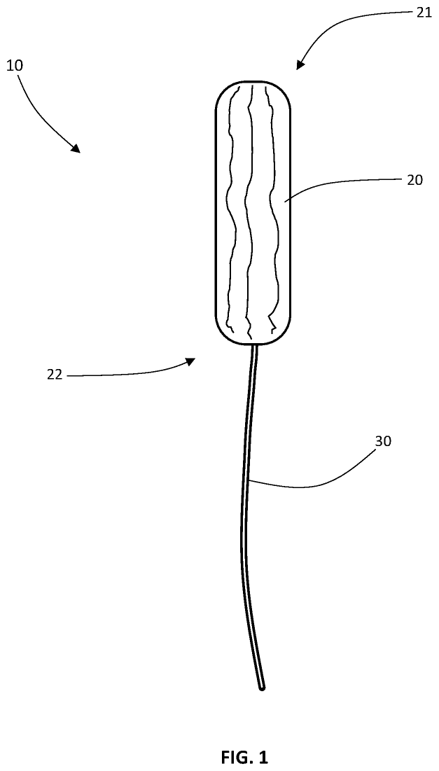

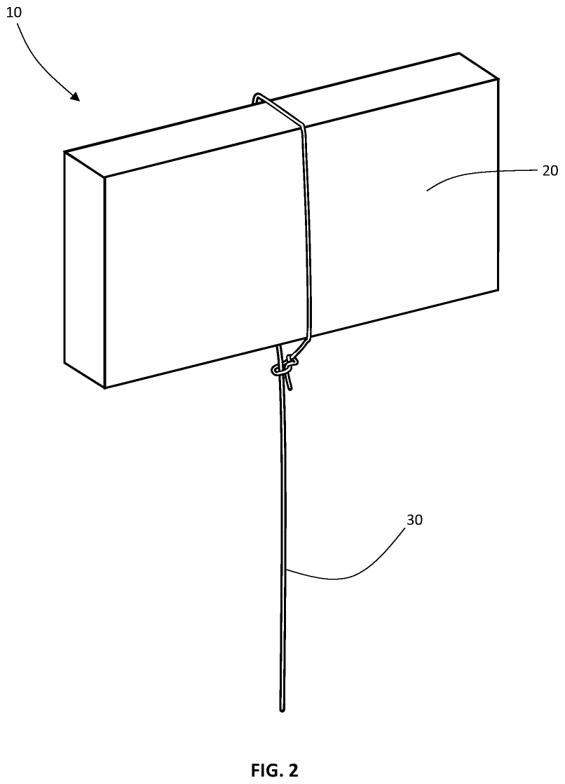

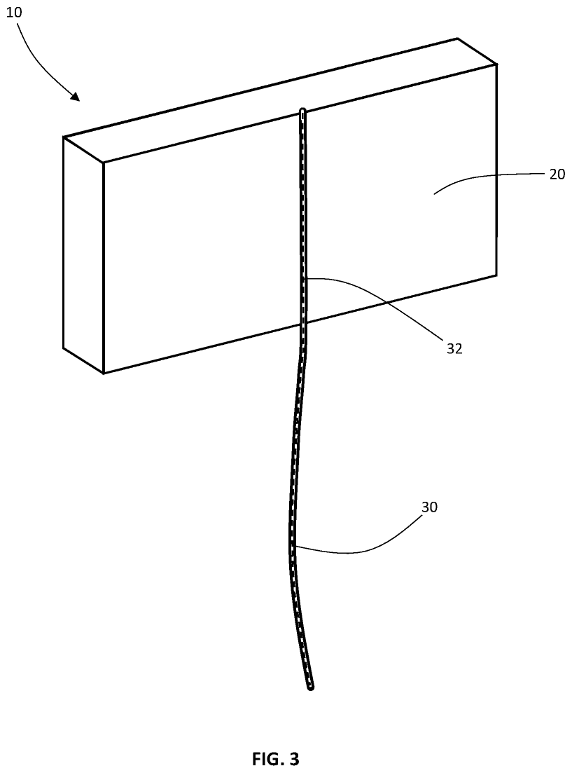

Referring to FIGS. 1-3, typically, a tampon 10 includes a pledget 20 and a withdrawal cord 30. The pledget 20 has a forward (insertion) end 21 and a rearward (withdrawal) end 22. The pledget 20 is manufactured of one or more absorbent materials, which have been assembled, cut, shaped and compressed in one or both of the lateral direction and the axial direction, in order to provide a compact, shape-stable body of a size and of form that, prior to encounter with fluid, facilitates easy insertion into a user's vaginal cavity. In some examples, the pledget is compressed laterally into a cylindrical and/or capsule-shaped form using a combination of physical mechanical compression and applied heating energy, which may include application of steam, to cause constituent fibers of the pledget to assume a “set” that enables the compressed pledget to retain its compressed form until the time it is applied by a user and is contacted by fluid. Components, assembly methods, and precompression configurations and shapes may vary among manufacturers and/or manufacturing processes. FIGS. 2 and 3 schematically illustrate an example of rectangular batt of absorbent material prior to compression.

The tampon may be provided new within an applicator assembly (not shown). When housed in an applicator assembly, the forward end 21 is proximate the forward (insertion) end of the barrel portion of the applicator assembly, proximate the petals (when included) of the barrel portion.

Preferably, a tampon will substantially retain its compressed shape and size as long as it is kept dry, prior to insertion and use. The pre-application shape and size need not persist following insertion and during use. Tampons typically expand in size and change shape in varying ways during use, as they are contacted by, absorb, and swell with absorbed fluid.

Tampons and pledgets also may be manufactured and formed to have other shapes and forms, as described in, for example, U.S. Pat. Nos. 6,824,536; 6,932,805; 8,597,267; 8,684,987; 8,216,202; 6,953,456; 6,554,814; 7,549,982; 6,939,340, and 8,029,485.

The pledget 20 may have a non-layered, uniform structure, or it may be a laminar structure comprised of integral or discrete layers. The pledget may have a folded structure, or it may be rolled, or have any other suitable physical structure and form known in the art. Generally, it may be preferred that the tampon have a certain minimal rigidity when new and dry and in its compressed form, to facilitate ejection from an applicator assembly and/or insertion, by pushing on the rearward end.

The pledget 20 may be constituted of a variety of liquid-absorbing fibrous materials commonly used in absorbent articles, such as, for example, rayon, cotton, or comminuted wood pulp (which is generally referred to as airfelt). Examples of other suitable absorbent materials include creped cellulose wadding; formations including meltblown filaments spun from polymeric resin, including coformed formations; chemically stiffened, modified or cross-linked cellulosic fibers; synthetic fibers such as crimped polyester fibers; peat moss; absorbent foams; tissue including tissue wraps and tissue laminates; or any other suitably absorbent material or combination or blend of absorbent materials. Preferred absorbent materials include cotton and rayon (including tri-lobal and conventional rayon fibers, and needle punched rayon). Suitable types of rayon may include GALAXY rayon (a tri-lobal rayon fiber structure) available as from Kelheim Fibres GmbH of Kelheim, Germany, and TENCEL rayon (a round fiber rayon) available from Lenzing AG, Lenzing, Austria. Suitable cotton material includes, long fiber cotton, short fiber cotton, cotton linters, T-fiber cotton, card strips, and comber cotton. Additionally, superabsorbent materials, such as superabsorbent polymers or absorbent gelling materials in particle or fiber form may be incorporated into the pledget structure.

The absorbent material(s) of the pledget 20 may be surrounded by an overwrap (not specifically shown). Such overwrap is preferably a liquid permeable material. Such materials may include knitted, woven or nonwoven fabrics of rayon, cotton, fibers spun from polymeric resins, including bicomponent fibers, or any other suitable natural or synthetic fibers known in the art. Rayon, polyethylene, polypropylene and blends of these are particularly suited for use as overwrap material. Synthetic fibers may include fibers spun or formed of polyester, polyolefin, nylon, polypropylene, polyethylene, polyacrylic, cellulose acetate, or combinations of these, such as bicomponent fibers. Natural fibers or semi-synthetic fibers may include fibers of cotton or rayon. In general, cotton and/or rayon fibers may be preferred for inclusion because they provide ready absorption and fluid wicking. Synthetic (polymer-based) fibers may also be preferred for inclusion for their surface characteristics and properties to balance the capillarity of more absorbent materials included, and enable the tampon to more readily slip against moist tissue, resulting in easier and more comfortable tampon insertion and removal. The overwrap may be adapted or formed to extend rearwardly beyond the rearward end of the assembly of absorbent material(s) to form a skirt portion, as described in U.S. Pat. No. 6,840,927, for example. The overwrap may be configured to extend from about 2 mm to about 30 mm beyond the rearward end 22 of the pledget.

The tampon contemplated herein includes a withdrawal cord 30, which may be attached to or along any portion of the pledget. In some examples the withdrawal cord 30 is attached to the pledget 20 by looping it around the pledget body. In some examples the withdrawal cord 30 is attached to the pledget by stitching 32, using a stitching thread.

The withdrawal cord may be formed from a cord, yarn or string stock having any suitable form known in the art, for example braided cord, knitted cord or twisted cord. A conventional type of withdrawal cord (in terms of thickness, material composition, etc.) may be periodically braided with a thicker slub of fibrous material, which acts as a wicking member, to form a structure to be connected to the remaining of the tampon. In such an embodiment, the portion of the cord, which will act as the withdrawal cord, may be treated to make it non-absorbent or even hydrophobic. It may also be a withdrawal cord as described in U.S. application Ser. No. 09/309,467.

The pledget may be provided with additional functional components, such as antimicrobial agents, lubricants, antioxidants, pH modifiers, etc., as known in the art.

It may be preferred in some circumstances the pledget 20 have the form of chevron-shaped laminar pad, prior to compression into a substantially cylindrical form. This pad may be formed to have a lateral width and a longitudinal length wherein the width is greater than the length. The pad may include one or more layers or batts of absorbent material, including an uppermost layer, a lowermost layer, and at least one intermediate layer positioned between the uppermost layer and lowermost layer. Each of the uppermost layer and the lowermost layer may be formed predominantly of rayon, and the at least one intermediate layer may be formed predominantly of cotton.

The tampon may be provided with an applicator assembly, and the tampon and/or the applicator assembly may have any structure, composition or features described in pending U.S. application Ser. No. 17/479,189.

Withdrawal Cord Composition and Features

The withdrawal cord typically is provided with a length sufficient for it to trail the rearward end of the pledget by a length sufficient for a free trailing end portion to protrude from the user's vagina when the pledget is fully inserted. When the cord is imparted with such a length, the user may easily grasp the free trailing end and pull the cord to withdraw the pledget from her body after a desired duration of use.

It is important that the withdrawal cord 30, while the tampon is in use, resist wicking of menstrual fluid. Users do not want menstrual fluid wicking down the cord to the portion protruding, or even to the trailing free end, because such wicking can create or increase the risk of soiling of underwear and outer clothing, with wicked fluid. Generally, it may be desired that a withdrawal cord material exhibit a Wicking Height no greater than 10 mm, more preferably no greater than 7 mm, and even more preferably no greater than 5 mm, when subjected to the Withdrawal Cord Wicking Height measurement method described below.

Menstrual fluid is, generally, aqueous. Accordingly, it may be desired that the withdrawal cord be constituted from fiber constituents that have hydrophobic surface chemistry, such that they do not draw flow of aqueous fluid along their surfaces. When the constituent fibers have hydrophobic surface chemistry, flow of aqueous fluid through interstitial passageways between constituent fibers of the cord structure (via capillary action) is not promoted.

Suitable plastic/polymeric resins are known and may be selected as components for spinning withdrawal cord fiber constituents which will inherently have suitably hydrophobic surfaces. Alternatively, or in combination, various petroleum-derived hydrophilizing surface treatment agents are known, which may be applied to fiber and/or cord surfaces, to render them hydrophobic or increase hydrophobicity.

As noted in the Background, however, uses of such plastics and/or treatment agents are becoming increasingly discouraged and disadvantageous in view of consumer preferences and regulatory pressures.

Consumer preferences for “natural” components for hygiene products has led some manufacturers of tampons to use withdrawal cord stock that is constituted predominantly (by weight) of natural plant fibers rather than spun polymer fibers. (Herein, “natural plant fibers” does not include fibers that are spun through spinnerets in manmade processes, e.g., from regenerated cellulose, for example, viscose, rayon, etc.) Suitable natural plant fibers may include cotton, jute, hemp, flax, ramie, and combinations thereof. Cotton fibers may be particularly preferred as withdrawal cord constituents for their physical characteristics including dry and wet tensile strength, amenability to yarn/thread spinning, pliability/softness, and proven human biocompatibility. With respect to the latter feature, cotton is widely used for medical products such as wound dressings/gauze/bandages. Currently, cotton is also in relatively abundant supply.

Following removal, by mechanical processing steps, of materials such as seeds, bits of leaves and stems, and relatively larger bits of foreign materials, cotton fiber intended for use in medical products or devices is further cleaned, purified and sanitized to remove materials including dirt particles, waxes, oils, deposits of environmental pollutants, pesticides, herbicides, pollens, yeasts, molds, bacteria and allergen proteins from the fibers. The cleaning process includes scouring in a caustic solution, followed by bleaching. Typically cotton fiber is scoured in a heated solution of water and sodium hydroxide, and bleached in a solution of hydrogen peroxide. These processes effectively remove the above-listed impurities and leave the cotton fiber with highly hydrophilic and absorbent properties, and a relatively bright white color. It has been believed that such steps are necessary to prepare the cotton fiber for use in medical products or devices. Additionally, the bright white color of such processed cotton has been preferred for personal hygiene products, because a relatively bright white color is an implicit signal of purity and sanitariness, for many consumers.

A cord stock that is braided, twisted or knitted from such conventionally processed cotton fiber thread or yarn will readily wick aqueous fluid. This makes it generally unsuitable as withdrawal cord stock, unless the manufacturer takes suitable mitigating steps.

Where a tampon manufacturer desires to provide a withdrawal cord constituted in part or in whole of cotton fiber, the current typical practice is to treat/impregnate the cord stock with a composition including one or more hydrophobizing agents. A composition including a suitable wax or blend of waxes may be used. For example, current PLAYTEX CLEAN COMFORT brand tampons (Edgewell Personal Care Company, Shelton, Connecticut, USA) are provided with a 100% cotton withdrawal cord. The cotton cord material is treated to add hydrophobizing agents included, in a blended composition, of paraffin, butyl stearate, carnauba wax, and acrylic polymer.

While such a composition can be effective at rendering the fibers of cotton withdrawal cord stock hydrophobic, and therefore, resistive to wicking of aqueous fluid, treating the cord stock with such composition adds processing steps, energy costs and material costs to the production of the cord stock. Further, the inclusion of paraffin (which itself is a very effective hydrophobizing agent) and acrylic polymer are counterproductive to the goal of reducing use/reliance on plastics and/or material components derived from petroleum.

It has been discovered, however, that a process for preparing cotton fiber withdrawal cord stock may be used that provides a relatively hydrophobic cord that resists wicking, does not include (or includes acceptably low levels) of impurities and molds, yeasts, bacteria and allergens, does not require further hydrophobizing treatment, and does not require addition of materials derived from petroleum.

Unprocessed cotton fiber includes natural waxes, produced by the cotton plants. These waxes render the cotton fibers in the bolls hydrophobic, thereby preventing them from absorbing water. Prior to harvesting, these waxes can cause the fibers in the open bolls to catch and hold airborne particulate contaminants (such as dust, pollen, yeasts and molds, etc.), which necessitates cleaning by scouring. It has been learned, however, that a scouring process that is relatively more gentle than conventional processes may be employed, that effectively removes these impurities and also allergen proteins, while leaving an effective portion of the natural cotton wax intact, on/within the fibers. As a result, the cotton fiber remains hydrophobic following processing, while still having been cleaned of objectionable impurities. A withdrawal cord constituted of such fiber has been discovered to be sufficiently resistive to wicking, for purposes contemplated herein.

As noted, typically, cotton fiber destined for medical product use is scoured in a heated solution of water and sodium hydroxide, with sodium hydroxide present in a concentration sufficient to impart a pH of 11 or higher, to the solution. It has been discovered, however that scouring cotton fiber (prior spinning into thread or yarn), or alternatively scouring cotton thread or yarn, or alternatively, scouring braided, twisted or knitted cord stock, in a heated solution having a pH greater than 7 but less than 11, can be effective to sufficiently remove objectionable impurities and allergens, while leaving behind a quantity of natural cotton wax with the fibers, sufficient to retain their hydrophobicity and resistance to wicking (measured as Wicking Height, as described herein). The lower-pH scouring solution may be provided by reducing the concentration of sodium hydroxide, or alternatively, by using relatively less-caustic agents such as sodium carbonate, sodium bicarbonate or combinations thereof. Sodium carbonate has been found to provide a particularly effective and efficient caustic solution for purposes contemplated herein.

Allowing an effective portion of the natural cotton wax to remain with the cotton fiber will leave the fiber and the withdrawal cord with an off-white, or slightly yellow, tan or beige hue. While it has been believed to date that consumers prefer a bright white color for items such as personal hygiene products, it has been discovered that consumers are increasingly accepting if not preferring an off-white coloration. Without intending to be bound by theory, it is believed that consumers have begun associating an off-white coloration with “natural” products, increasingly preferred over products perceived as artificially or synthetically produced or constituted.

With respect to chemical composition, the natural wax material present in unprocessed cotton is relatively complex. Methods for identifying its components and characterizing its composition and constituent concentrations are complicated. It has been discovered, however, that measures of the hue/coloration of a sample of withdrawal cord correlate, relatively, with the quantities of natural wax present. Accordingly, to provide a cord constituted predominantly, substantially entirely or entirely of cotton fibers (by weight) that is suitably hydrophobic, while also having had objectionable contaminants removed to a sufficient degree, it may be desired that the cord exhibit one or any combination of the following ranges of measured values, when measured using the Withdrawal Cord Color Measurement described below:
Whiteness Index of 14 to 20;
Yellowness Index of 20 to 23;
Delta E (1976) of 12 to 14; and
b* value of 6 to 12.

It may be appreciated that a manufacturer may seek to impart a color or hue to withdrawal cord stock by artificial means, i.e., by means other than controlling the amount of natural wax allowed to remain in the cotton fiber following processing. For example, a manufacturer may dye the cotton fiber either concurrently with, or following, the scouring or bleaching step, and then add hydrophobizing wax to the scoured, or bleached, and dyed cotton fiber in a subsequent treatment step. Additionally, or alternatively, the manufacturer may add wax or other surface coating in a treatment step following scouring or bleaching, where the added wax or surface coating has its own natural or artificially-imparted off-white coloration, and imparts that coloration to the cord fiber upon treatment. However, it is contemplated herein that the ranges of color values set forth above are imparted by the natural color of the fiber and presence of natural cotton wax left behind following the scouring process, and not by a dyeing process following scouring or bleaching or addition of wax or other surface coating following scouring or bleaching.

For purposes contemplated herein, it may be desired to spin the constituent yarn or thread, or even to manufacture the complete withdrawal cord stock material (such as by braiding, knitting, or twisting multiple yarn strands), from the constituent cotton fiber, prior to scouring. This is because the natural waxes present and relatively well- and evenly-distributed on and/or within the unscoured cotton fibers serve as lubricants and processing aids in the mechanical spinning, braiding or knitting processes, reducing or eliminating a need to artificially add lubricants to the fiber, and thereby simplifying the cord stock manufacturing process. Accordingly, the constituent spun yarn or thread, or even the entirety of the cord stock in its structurally complete form (e.g., twisted, knitted or braided), may be subjected to the relatively gentle scouring process contemplated herein.

In some examples, the constituent cotton fiber, the constituent cotton fiber yarn or thread, or the structural entirety of the manufactured withdrawal cord stock, may be scoured in a heated aqueous solution of sodium carbonate, in a concentration sufficient to impart the solution with a pH of greater than 7, to less than 11, more preferably 8 to 10, and still more preferably 8.5 to 9.5. The solution may be heated to near boiling, e.g., a temperature of at least 82° C., more preferably at least 88° C., even more preferably at least 93° C., and still more preferably at least 97° C. The cotton material may be skeined, spooled, wound, etc., and placed in a vessel (e.g., a kier), in which the heated scouring solution is agitated and/or circulated so as to be caused to flow across and through the fiber structures. It has been found that a scouring time of approximately 15 to 75 minutes, or approximately 20 to 70 minutes, or even approximately 25 to 65 minutes, or even approximately 30 to 60 minutes, may be effective for purposes contemplated herein.

Any one or any combination of pH of the aqueous scouring solution, temperature of the aqueous scouring solution, duration/time of scouring process, and agitation level and/or flow velocity of the aqueous scouring solution through/across the fibers, may be adjusted to effect desirably low Wicking Height and measured color features (measured as described herein) of the withdrawal cord stock, as described herein.

In some examples, the withdrawal cord may be affixed to the pledget via stitching, and stitching thread. In some examples, the stitches may be applied and be present along a portion, majority or substantially the entirety of the trailing length of the withdrawal cord, as a result of the tampon manufacturing and stitching processes. In such examples, it may be deemed desirable that the stitching thread also resist wicking. In such examples, the stitching thread may be manufactured of cotton fiber and in a manner similar to that described above, for withdrawal cord stock. Alternatively, the stitching thread may be manufactured by different methods and/or of different components, but still including processing steps and/or thread constituents combined to produce a stitching thread that is hydrophobic and resists wicking.

### Measurement Methods

Withdrawal Cord Wicking Height

For purposes herein, the extent to which an example of tampon withdrawal cord tends to wick aqueous fluid is measured by submerging a specified, vertically oriented length of a specimen of the cord into an aqueous solution containing dye, for four hours, and then measuring the height the fluid has wicked upwardly in the non-submerged portion of the cord, as reflected by staining of the cord specimen by the dye. All measurements are performed in a laboratory maintained at 23° C.±2° C. and 50%±2% relative humidity.

The measurement specimens are prepared as follows. Clean, new disposable exam-grade medical gloves must be worn while preparing the test specimens and during the measurement procedure in order to prevent contamination from contact with the analyst's hands.

For a cord that is connected to a finished tampon product, remove the tampon from its wrapper, then gently expel the tampon from any applicator present. For a connected cord, the test specimen is the cord while it remains connected to the tampon pledget. Thus, it is not necessary to cut or detach the cord from the pledget.

It is understood that some tampon products have withdrawal cords that are stitched to the pledgets with stitching thread that is distinct from the withdrawal cord stock, and in some such examples, the stitching may be present along a portion or even majority of the length of the withdrawal cord. The stitching thread may have a composition differing from that of the withdrawal cord. For such examples:
For purposes of claims herein that do not make any reference to stitching thread or Wicking Height of stitching thread: Using fine pointed scissors, carefully snip the stitches to the extent necessary (without snipping any portion of the cord) and gently remove them from the portion of the cord that extends free of the pledget.
For purposes of claims herein that recite in substance that the withdrawal cord is stitched to the pledget with stitching thread, and that the stitching thread Wicking Height is no greater (by a specified margin) than the withdrawal cord Wicking Height, the stitches are to be left intact in the withdrawal cord, and the trailing end portion of the withdrawal cord including the stitches are to be submersed in the test fluid, in application of the measurement method described herein.

It is understood that some tampon products have a withdrawal cord formed of a strand of cord stock that has been looped around the pledget, or looped through a hole in the pledget, and then doubled to constitute a trailing portion of the withdrawal cord, the two portions of the strand constituting the doubled portion being knotted or otherwise affixed together proximate the trailing ends of the portions. For such examples, both portions of the strand are deemed to constitute the withdrawal cord, and both are to be submerged in test fluid together in the condition in which the tampon was removed from the wrapper and applicator, in application of the measurement method described herein.

For unconnected cord stock, the test specimen is a length of cord stock cut to a length of approximately 240 mm, obtained from a clean portion of example cord stock that has not been previously handled by humans, in a condition in which it would be expected to be combined with a pledget to form a finished tampon product.

A total of three test specimens are prepared, for each cord example to be analyzed. The test specimens are equilibrated in a room maintained at 23° C.±2° C. and 50%±2% relative humidity for 2 hours prior to testing.

The test fluid is prepared by adding 10.0 grams±0.5 grams of sodium chloride (reagent grade, any convenient source) and 0.10 grams±0.01 grams of methylene blue powder (CAS #61-73-4, any convenient source) to a 1-liter volumetric flask. Now add a sufficient quantity of deionized water to the fill line of the flask, and agitate until both reagents are completely dissolved.

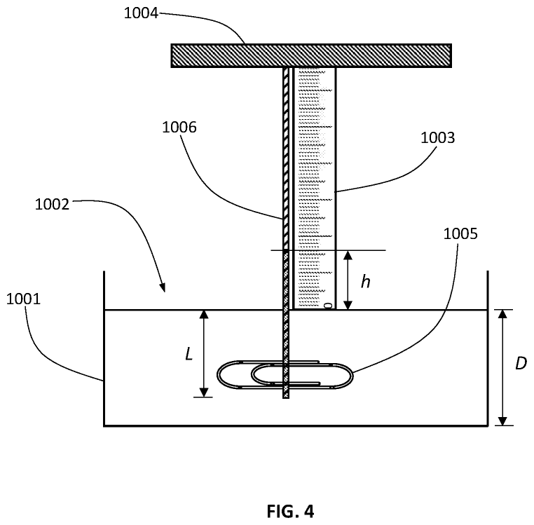

The test apparatus and arrangement is depicted in FIG. 4. The test apparatus includes a transparent dish 1001 filled to a depth D, of 50 mm with the prepared colored test fluid. The filled dish is placed on a horizontally level benchtop. A steel metric ruler 1003 (traceable to NIST, or equivalent) is secured to a rigid support 1004 such that the ruler 1003 is oriented vertically above the test fluid surface 1002. A small weight 1005 (about 2 grams; such as a large paper clip or equivalent) is secured to the trailing end portion of the prepared test specimen 1006. The leading end (or tampon pledget) of the test specimen 1006 is then secured to a rigid support 1004 such that the specimen is oriented vertically above the test fluid and positioned adjacent to the ruler 1003. Slowly lower the weighted, trailing end of the test specimen 1006 into the test fluid such that a length, L, of the test specimen is submerged beneath the test fluid surface 1002. Length L is at least 25 mm but not more than 45 mm. Now adjust the ruler 1003 such that its zero edge or zero marker is positioned at exactly the main level of the test fluid surface 1002 (i.e., not the level of the fluid that may be higher or lower where it meets the ruler, as a result of test fluid surface tension and hydrophilicity or hydrophobicity of the ruler surface). Leave the test specimen submerged and undisturbed for a period of 4 hours. During the test, the test fluid that travels by wicking upwardly into the non-submerged portion of the test specimen 1006 will leave a visible stain on the test specimen as a result of the methylene blue dye content.

After 4 hours have expired, use the ruler to measure the height h, of the uppermost extent of the stain on the cord stock. Record the stain height h, to the nearest millimeter.

Repeat the procedure for a total of three replicate test specimens. Calculate the arithmetic mean for stain height h for all three replicate test specimens and report the result as Wicking Height, to the nearest millimeter.

It is understood, and it may be observed in some examples, that the solvent water and/or NaCl solution may wick farther up a withdrawal cord specimen than the uppermost extent of methylene blue dye staining. Interaction with the cotton or other cord stock constituents may cause the dye molecules to come out of solution and attach to the fibers, while the remaining components of the test fluid continue to wick upwardly through the cord. Accordingly, it is understood that the results of this method will be a reflection of the relative wicking capability of the cord material, rather than an absolute measurement.

Withdrawal Cord Color Measurement

The CIE yellowness and whiteness indices for tampon cords are calculated from instrumentally measured X, Y, and Z color coordinate values in accordance with compendial method ASTM E313-20 using the test parameters described herein. The total color difference (Delta E*1976) between the tampon cord and a “standard white” is calculated from instrumentally measured L*a*b* color coordinate values obtained for the tampon cord using the test parameters described herein. The CIE b* value is measured and reported as an additional indication of the yellowness of the tampon cord. Color analyses are made using a spectrophotometer that has 0°/45° circumferential geometry with adjustable apertures (e.g. port plate openings) capable of making standard CIE L*a*b* and CIE tristimulus X, Y, and Z measurements in accordance with ASTM E1349. An example of a suitable spectrophotometer is the Labscan XE (available from Hunter Associates Laboratory, Inc., Reston, VA, or equivalent). To ensure a consistent and uniform black color, the matte side of a PANTONE “black onyx” plastic standard chip (color designation PQ-19-4003TCX) (Pantone LLC/X-Rite, Incorporated, Grand Rapids, Michigan, USA) is used as the sample backing material. All testing is performed in a room maintained at a temperature of 23° C.±2.0° C. and a relative humidity of 50%±2% and samples are conditioned under the same environmental conditions for at least 2 hours prior to testing.

Test samples are prepared by securing them to the black PANTONE plastic standard chip as follows. Withdrawal cord stock that has not yet been combined with a pledget to form a finished tampon is the ideal source for samples, however, cords removed from finished tampons may also be used. If the cord is obtained from a finished tampon, the cord is cut away from the tampon pledget and any distinct, adjacently disposed materials constituting the tampon, as well as any stitching thread that might be present is carefully removed from the cord material.

A sufficient number of separate cords are required to prepare a sampling area that will entirely cover the opening on the port plate of the testing instrument (0.50 inch diameter opening). To prepare the test sample, secure one end of the cord to the back (i.e. glossy) side of the black PANTONE plastic standard chip. Now, keeping the cord gently taut, wind it around the chip to cover the matte black side of the chip in successive rows, excluding the label and hole near the top of the chip (dimensions of area to be covered by the cord are about 40 mm by 48 mm). The cord must be wrapped without slack such that each wound row of cord is gently taut but not destructively stretched, and each wound row of cord is urged against its precedingly wound neighboring row, to prevent any portion of the black chip from being visible between the rows of cord. Secure the trailing end of the wrapped cord to the back (glossy) side of the black chip. The properly prepared test sample will be a chip having a single layer of consecutively wound rows of cord that present a generally macroscopically flat surface, with no portion of the black chip visible between the rows. This preparation will provide enough surface area to allow for five replicate measurements to be made. If individual cords from finished tampons are to be tested (rather than a continuous length of cord stock) and continuous winding of cord about the chip to provide the required test surface area cannot be had, then each end of each individual sample cord is secured to the back (glossy) side of the black chip, with each cord wrap being urged against its neighboring cord in such a way that no portion of the black chip is visible between the individually secured cords. Attach a sufficient number of individual cords to cover the matte black portion of the chip (about 40 mm by 48 mm).

To measure color, calibrate and standardize the instrument per the vendor instructions using the standard white and black tiles provided by the vendor. Set the spectrophotometer to report CIE L*a*b* and CIE X, Y, and Z color coordinates with a D65 standard illumination, a 10° observer, a 0.40 inch area view, a 0.50 inch aperture, and the UV filter set to nominal. If the testing instrument cannot accommodate the simultaneous collection of L*a*b* and X, Y, Z color coordinates, then these two sets of coordinates are to be collected in separate steps. Place the prepared test sample over the opening in the port plate such that the entire opening is covered by the cord material on the matte black side of the plastic chip. Ensure that only the matte black portion of the plastic chip that is fully covered by the cord is within the port opening (i.e. any tape or other means of securing the cord to the chip is avoided). Press the prepared sample over the port opening and take a reading. Record the L*a*b* and X, Y, and Z values to the nearest 0.01 units. Repeat for a total of five replicate measurements of differing areas of the chip. To note, replicates can be measured on the previously prepared test sample as long as each replicate is obtained from non-overlapping portions of the test sample.

Use the Y value from the measured X, Y, and Z color coordinates to calculate CIE yellowness index (YI) for each replicate in accordance with ASTM E313-20 as follows, and record as YI to the nearest 0.01 units.

YI=[100*(CₓX−C_{z}Z)]/Y
where CₓX=1.3013 and C_{z}Z=1.1498 (specific for D65 illuminant and 10 degree observer)

To calculate the CIE whiteness index in accordance with ASTM E313-20, the chromaticity coordinates x and y of the CIE XYX color space must be obtained. Use the measured X, Y, and Z color coordinate values to calculate x and y for each replicate measurement as follows.

x=X/(X+Y+Z)

y=Y/(X+Y+Z)

Now calculate CIE whiteness index (WI) for each replicate in accordance with ASTM E313-20 as follows, and record as WI to the nearest 0.01 units.

WI=Y+(WI,ₓ)*(xₙ−x)+(WI,_{y})*(yₙ−y)

where WI,ₓ=800, xₙ=0.3138, WI,_{y}=1700 and yₙ=0.331 (specific for D65 illuminant and 10 degree observer)

The total color difference (Delta E* 1976) between the tampon cord test sample and a “standard white” is calculated using the measured L*a*b* color coordinate values for the tampon cord test sample and defining our “standard white” to be used herein as having color coordinate values of L*=96.34, a*=−1.2 and b*=2.27, equivalent to that of PANTONE “bright white” (color designation 11-0601 TCX) (Pantone LLC/X-Rite, Incorporated, Grand Rapids, Michigan, USA). The following equation is used to calculate Delta E* (1976) for each replicate measurement, where Delta E* is recorded to the nearest 0.1 units:

Delta E*(1976)=√[(L₂*−L₁*)²+(a₂*−a₁*)²+(b₂*−b₁*)²]

where L₁*, a₁* and b₁* are those values assigned to our “standard white” as defined herein and L₂*, a₂* and b₂* are the measured color coordinate values for each replicate measurement

The arithmetic mean for yellowness index, whiteness index, b* and Delta E* is calculated across all five replicate measurements and reported as YI to the nearest 0.01 units, WI to the nearest 0.01 units, b* to the nearest 0.01 units and Delta E* to the nearest 0.1 units.

In view of the disclosure above, the following non-limiting examples are contemplated:
1. A tampon comprising an absorbent pledget and a withdrawal cord attached to the pledget, wherein the withdrawal cord is constituted predominantly (by weight) of natural plant fiber, comprises no more than 5 percent by weight material derived from petroleum, and exhibits a Wicking Height no greater than 10 mm, more preferably no greater than 7 mm, and even more preferably no greater than 5 mm.
2. The tampon of example 1 wherein the withdrawal cord is constituted predominantly (by weight) of fiber selected from the group consisting of cotton, jute, hemp, flax, ramie, and combinations thereof, more preferably predominantly cotton, and even more preferably greater than 90 percent by weight cotton together with materials typically present in ginned/mechanically cleaned but chemically unprocessed cotton.
3. The tampon of either of examples 1 or 2 wherein the withdrawal cord comprises no more than 5 percent by weight material that is not inherently present in the natural plant fiber following harvesting but prior to scouring.
4. The tampon of any of the preceding examples wherein the natural plant fiber has been scoured in an aqueous solution having a pH of greater than 7 to less than 11, more preferably 8 to 10, and still more preferably 8.5 to 9.5.
5. The tampon of example 4 wherein the aqueous solution comprises a compound selected from the group consisting of sodium hydroxide, sodium carbonate, sodium bicarbonate and combinations thereof, and preferably sodium carbonate.
6. The tampon of any of the preceding examples wherein the withdrawal cord exhibits any one or any combination of the following ranges of measured values:
   Whiteness Index of 14 to 20;
   Yellowness Index of 20 to 23;
   Delta E (1976) value of 12 to 14; and/or
   b* value of 6 to 12.
7. The tampon of example 6 wherein the recited Whiteness Index, Yellowness Index, Delta E (1976) value and/or b* value is/are imparted to the withdrawal cord by inherent coloration of the fiber and/or material in or on the fiber, left behind following scouring.
8. The tampon of any of the preceding examples wherein the withdrawal cord is stitched to the pledget with stitching thread, and stitching (32) is present along a portion of the trailing length of the withdrawal cord proximate a trailing end thereof, and the Wicking Height measured on the stitching thread is not greater than the Wicking Height measured on the withdrawal cord, by an amount greater than 20 percent of the Wicking Height measured on the withdrawal cord.
9. A process for manufacturing tampons (10), comprising a sub-process for manufacturing tampon withdrawal cord stock, the withdrawal cord stock manufacturing sub-process comprising the steps of:
   providing constituent cotton fiber, and
   scouring the cotton fiber, either prior to or following spinning of the cotton fiber into yarn or thread, in a heated aqueous solution having a pH of greater than 7, to less than 11, more preferably 8 to 10, and even more preferably 8.5 to 9.5.
10. The method of example 9, further comprising the step of spinning the constituent cotton fiber into yarn or thread, prior to the scouring step.
11. The method of either of examples 9 or 10, further comprising the step of twisting, braiding or knitting yarn or thread constituted at least partially of the cotton fiber, prior to the scouring step.
12. The method of any of examples 9-11, wherein the aqueous solution comprises sodium hydroxide, sodium carbonate, sodium bicarbonate or any combination thereof, and preferably sodium carbonate.
13. The method of any of examples 9-12 wherein one or more of the aqueous solution pH, aqueous solution temperature, and scouring time are adjusted so as to result in withdrawal cord stock exhibiting a Wicking Height no greater than 10 mm, more preferably no greater than 7 mm, and even more preferably no greater than 5 mm.
14. The method of any of examples 9-13, wherein one or more of the aqueous solution pH, aqueous solution temperature, and scouring time are adjusted so as to result in withdrawal cord stock exhibiting any one or any combination of the following ranges of measured values:
   Whiteness Index of 14 to 20;
   Yellowness Index of 20 to 23;
   Delta E (1976) value of 12 to 14; and/or
   b* value of 6 to 12.
15. The tampon of example 14 wherein the recited Whiteness Index, Yellowness Index, Delta E (1976) value and/or b* value is/are imparted to the withdrawal cord by inherent coloration of the constituent cotton fiber and/or natural wax on the constituent cotton fiber, left behind following scouring.
16. The method of any of examples 9-15, wherein no step or combination of steps is/are included that add more than, by weight, 5 percent, more preferably 3 percent, even more preferably 1 percent, and still more preferably substantially 0 percent, material comprising or derived from petroleum, to the withdrawal cord stock.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “approximately 40 mm.”

Where ranges of measured quantities or property values are described and/or recited as characterizations of subject matter contemplated herein, such ranges are deemed to include and contemplate any and all sub-ranges within the ranges.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

In view of the description above, the following non-limiting examples are contemplated. Any of these examples as well as others may be claimed in one or more subsequent non-provisional patent applications based in whole or in part on the disclosure herein:

What is claimed is:

1. A tampon comprising an absorbent pledget and a withdrawal cord attached to the pledget, wherein the withdrawal cord is constituted predominantly (by weight) of natural plant fiber, comprises no more than 5 percent by weight material derived from petroleum, and exhibits a Wicking Height no greater than 10 mm.

2. The tampon of claim 1 wherein the withdrawal cord is constituted predominantly (by weight) of fiber selected from the group consisting of cotton, jute, hemp, flax, ramie, and combinations thereof.

3. The tampon of claim 1 wherein the withdrawal cord comprises no more than 5 percent by weight material that is not inherently present in the natural plant fiber following harvesting but prior to scouring.

4. The tampon of claim 1 wherein the natural plant fiber has been scoured in an aqueous solution having a pH of greater than 7 to less than 11.

5. The tampon of claim 4 wherein the aqueous solution comprises a compound selected from the group consisting of sodium hydroxide, sodium carbonate, sodium bicarbonate and combinations thereof.

6. The tampon of claim 1 wherein the withdrawal cord exhibits any one or any combination of the following ranges of measured values:
Whiteness Index of 14 to 20;
Yellowness Index of 20 to 23;
Delta E (1976) value of 12 to 14; and/or
b* value of 6 to 12.

7. The tampon of claim 6 wherein the recited Whiteness Index, Yellowness Index, Delta E (1976) value and/or b* value is/are imparted to the withdrawal cord by inherent coloration of the fiber and/or material in or on the fiber, left behind following scouring.

8. The tampon of claim 1 wherein the withdrawal cord is stitched to the pledget with stitching thread, and stitching is present along a portion of the trailing length of the withdrawal cord proximate a trailing end thereof, and the Wicking Height measured on the stitching thread is not greater than the Wicking Height measured on the withdrawal cord, by an amount greater than 20 percent of the Wicking Height measured on the withdrawal cord.

9. A process for manufacturing tampons, comprising a sub-process for manufacturing tampon withdrawal cord stock, the withdrawal cord stock manufacturing sub-process comprising the steps of:
providing constituent cotton fiber, and
scouring the cotton fiber, either prior to or following spinning of the cotton fiber into yarn or thread, in a heated aqueous solution having a pH of greater than 7, to less than 11.

10. The method of claim 9, further comprising the step of spinning the constituent cotton fiber into yarn or thread, prior to the scouring step.

11. The method of claim 9, further comprising the step of twisting, braiding or knitting yarn or thread constituted at least partially of the cotton fiber, prior to the scouring step.

12. The method of claim 9, wherein the aqueous solution comprises sodium hydroxide, sodium carbonate, sodium bicarbonate or any combination thereof.

13. The method of claim 9 wherein one or more of the aqueous solution pH, aqueous solution temperature, and scouring time are adjusted so as to result in withdrawal cord stock exhibiting a Wicking Height no greater than 10 mm.

14. The method of claim 9, wherein one or more of the aqueous solution pH, aqueous solution temperature, and scouring time are adjusted so as to result in withdrawal cord stock exhibiting any one or any combination of the following ranges of measured values:
Whiteness Index of 14 to 20;
Yellowness Index of 20 to 23;
Delta E (1976) value of 12 to 14; and/or
b* value of 6 to 12.

15. The tampon of claim 14 wherein the recited Whiteness Index, Yellowness Index, Delta E (1976) value and/or b* value is/are imparted to the withdrawal cord by inherent coloration of the constituent cotton fiber and/or natural wax on the constituent cotton fiber, left behind following scouring.

16. The method of claim 9, wherein no step or combination of steps is/are included that add more than, by weight, 5 percent material comprising or derived from petroleum, to the withdrawal cord stock.
